(19) United States
(12) Patent Application Publication
(10) Pub. No.: US 2007/0083228 A1
(43) Pub. Date: Apr. 12, 2007

(54) Multi-pronged biopsy grasping jaws

(75) Inventors: Francesco Visinoni, Mozzo (IT); Jim Milios, Azzonica (IT)

Correspondence Address: KENYON & KENYON LLP, 1500 K STREET N.W., SUITE 700, WASHINGTON, DC, 20005, US

(73) Assignee: Milestone s.r.I., Sorisole (IT)

(21) Appl. No.: 11/247,150

(22) Filed: Oct. 12, 2005

(51) Int. Cl. A61B 17/50

(52) U.S. Cl. 606/210

(57) ABSTRACT

The present invention relates to a grossing tool for preparing slices of tissue having a uniform predetermined thickness. The tool comprises a first and a second jaw for holding a tissue sample in a substantially fixed position therebetween. The first jaw and the second jaw have a first and a second array of prongs, respectively, for engaging the tissue sample. Furthermore, the prongs have parallel edges for guiding a cutting tool and form at least a cutting slot of a predetermined width corresponding to a predetermined thickness of a slice to be cut from the tissue sample. An actuator moves the first jaw and the second jaw in a plane of movement oriented perpendicular to the first and the second array of prongs between an open position and a holding position for holding the tissue sample. The grossing tool enables even an inexperienced user to cut tissue samples having a predetermined and substantially uniform thickness.

### FIELD OF THE INVENTION

This invention relates to tools for preparing tissue samples, and in particular to a grossing tool for preparing slices of tissue samples having a uniform predetermined thickness.

### BACKGROUND OF THE INVENTION

In the field of pathology, it is customary to prepare slice-shaped tissue samples for processing, staining, and histopathologic analysis. One practice of examining fresh or fixed patient tissue samples is referred to as “grossing” and involves preparation of relatively thick tissue samples, i.e. samples in the order of 0.5 to 4 mm thickness are considered the norm.

The standardization of cassettes of an internal limit of 5 mm thickness is considered to be the physical limit of tissue thickness for routine histoprocessing. However, squeezing of tissue is occurring frequently, resulting in compression marks on the tissue from the cassette and its cover.

During the grossing process representative tissue sample slices are taken from various sites of the tissue for subsequent histoprocessing steps such as: fixing in formalin, dehydrating, clearing and ultimately impregnating in paraffin wax. This enables paraffin sections to be cut and stained for microscopical examination and diagnosis.

The thickness and uniformity of the tissue sample slice determine histoprocessing time and quality of the histoprocessed tissue sample slice for diagnosis. The current practice in routine grossing is to rely on the skill of an experienced pathologist or pathology assistant for estimating the tissue sample slice thickness and maintaining its uniform thickness during the cutting process. This process is done manually which involves grasping the tissue with a pair of forceps at a distal end, holding the tissue down at the point of contact with the forceps, and then cutting. Alternatively, the tissue is sandwiched between single pronged forceps or fingers. The pathologist then cuts through the tissue without any physical guide for ensuring a desired thickness and its uniformity. As is evident, this practice normally does not produce tissue sample slices of a predetermined uniform thickness. The process is further complicated when cutting very soft tissues in a fresh state or fatty tissue, which are extremely difficult to cut to a predetermined thickness even for the most experienced pathologist.

Lack of uniformity in the thickness of a tissue sample block results in poor histoprocessing rendering a subsequent microscopic diagnosis difficult if not impossible. It is, therefore, essential to cut a tissue sample slice having a uniform—preferably less than 3 mm—thickness in order to ensure consistent histoprocessing quality that does not jeopardize subsequent microscopic diagnosis.

In U.S. Pat. No. 6,513,803 issued Feb. 04, 2003, Morales et al. disclose a grossing board having one or more tissue receiving depressions formed therein. A knife guiding assembly is provided to guide a knife or scalpel blade along a surface of the grossing board to cut the tissue sample to a predetermined thickness defined by the depth of the respective depression. While the grossing board facilitates the preparation of some tissue samples, tissues that are very soft, smooth and slippery, such as those having a high fat content, are difficult to hold in the receiving depressions of the grossing board. Furthermore, the receiving depressions are difficult to clean resulting in an increased risk of cross contamination between different samples.

In U.S. Patent Application published Apr. 01, 2004 as US 2004/0061269, Morales discloses a pathology grossing tool comprising first and second tine arrays for engaging and holding the tissue. The first and second tine arrays define a slice thickness therebetween when a slice is prepared by cutting parallel to the first and second tine array using the tine arrays as guides for a cutting tool. Unfortunately, by engaging the tissue with the tine array the tissue structure is distorted in the vicinity of the tines and, further, the tissue is compressed between the tines resulting in an uneven slice thickness after cutting. Furthermore, improper engagement of the tines with the tissue, in particular, towards the end of the cutting process, allows movement of the tissue sample due to shearing stress induced by the moving cutting tool also resulting in an uneven thickness of the sample slice.

It would be advantageous to overcome the drawbacks of the prior art by providing a grossing tool that enables cutting of slices of various tissue samples having a uniform slice thickness. It would be further advantageous to provide a grossing tool that is easy to handle, easy to clean and easily portable for use in a variety of situations.

### SUMMARY OF THE INVENTION

It is, therefore, an object of the invention to provide a grossing tool that overcomes some of the drawbacks of the prior art.

In accordance with the present invention there is provided a grossing tool comprising: a first and a second jaw for holding an object in a substantially fixed position therebetween, the first jaw and the second jaw having a first and a second array of prongs, respectively, for engaging the object, the prongs of each of the first and the second array of prongs having parallel edges for guiding a cutting tool, the parallel edges forming at least a cutting slot of a predetermined width corresponding to a predetermined thickness of a slice to be cut from the object, the at least a cutting slot of the first array of prongs being placed opposite the corresponding at least a cutting slot of the second array of prongs; and, an actuator in mechanical communication with the first jaw and the second jaw for moving the first jaw and the second jaw in a plane of movement oriented perpendicular to the first and the second array of prongs between an open position and a holding position for holding the object.

In accordance with the present invention there is further provided a grossing tool comprising:

a U-shaped band of elastic material having at an end of a first arm a first jaw and at an end of a second arm a second jaw for holding an object in a substantially fixed position therebetween, the first jaw and the second jaw having a first and a second array of prongs, respectively, for engaging the object, the prongs of each of the first and the second array of prongs having parallel edges for guiding a cutting tool, the parallel edges forming at least a cutting slot of a predetermined width corresponding to a predetermined thickness of a slice to be cut from the object, the at least a cutting slot of the first array of prongs being placed opposite the corresponding at least a cutting slot of the second array of prongs, the first jaw and the second jaw being movable in a plane of movement oriented perpendicular to the first and the second array of prongs between an open position and a holding position for holding the object.

In accordance with the present invention there is yet further provided a grossing tool comprising:

a first member having at a first end a first jaw and at a second opposite end a first handle and a second member having at a first end a second jaw and at a second opposite end a second handle, the first jaw and the second jaw for holding an object in a substantially fixed position therebetween, the first and the second jaw having a first and a second array of prongs, respectively, for engaging the object, the prongs of each of the first and the second array of prongs having parallel edges for guiding a cutting tool, the parallel edges forming at least a cutting slot of a predetermined width corresponding to a predetermined thickness of a slice to be cut from the object, the at least a cutting slot of the first array of prongs being placed opposite the corresponding at least a cutting slot of the second array of prongs; and, a pivot for connecting the first member and the second member and for moving the first jaw and the second jaw in a plane of movement oriented perpendicular to the first and the second array of prongs between an open position and a holding position for holding the object.

### BRIEF DESCRIPTION OF THE FIGURES

Exemplary embodiments of the invention will now be described in conjunction with the following drawings, in which:

FIGS. 1a and 1b schematically illustrate a side view and a top view of a grossing tool according to the invention;

FIGS. 1c and 1d schematically illustrate a cross sectional view and a side view of a detail of the grossing tool according to the invention while in operation;

FIGS. 1e and 1f schematically illustrate a top view of an embodiment of a grossing tool according to the invention;

FIGS. 2a to 2c schematically illustrate in a top view various shapes of jaw plates used in the grossing tool according to the invention;

FIGS. 3a to 3f schematically illustrate in a top view various of prongs and cutting slots for use with the grossing tool according to the invention;

FIG. 4 schematically illustrates in a top view various dimensions of the prongs and cutting slots for use with the grossing tool according to the invention;

FIGS. 5a to 5e schematically illustrate in top views and cross sectional views various embodiments of a gripping surface for use with the grossing tool according to the invention;

FIG. 6 schematically illustrates a top view of another embodiment of a grossing tool according to the invention;

FIG. 7a schematically illustrates a top view of yet another embodiment of a grossing tool according to the invention;

FIGS. 7b and 7c schematically illustrate a side view and a top view of yet another embodiment of a grossing tool according to the invention;

FIGS. 7d and 7e schematically illustrate a top view and a cross-sectional view of yet another embodiment of a grossing tool according to the invention;

FIGS. 8a and 8b schematically illustrate a top view of yet two other embodiments of a grossing tool according to the invention;

FIGS. 9a to 9c schematically illustrate a top view yet another embodiment of a grossing tool according to the invention; and,

FIGS. 10a to 10d schematically illustrate in a side view various orientations of the prongs of the grossing tool according to the invention.

### DETAILED DESCRIPTION OF THE INVENTION

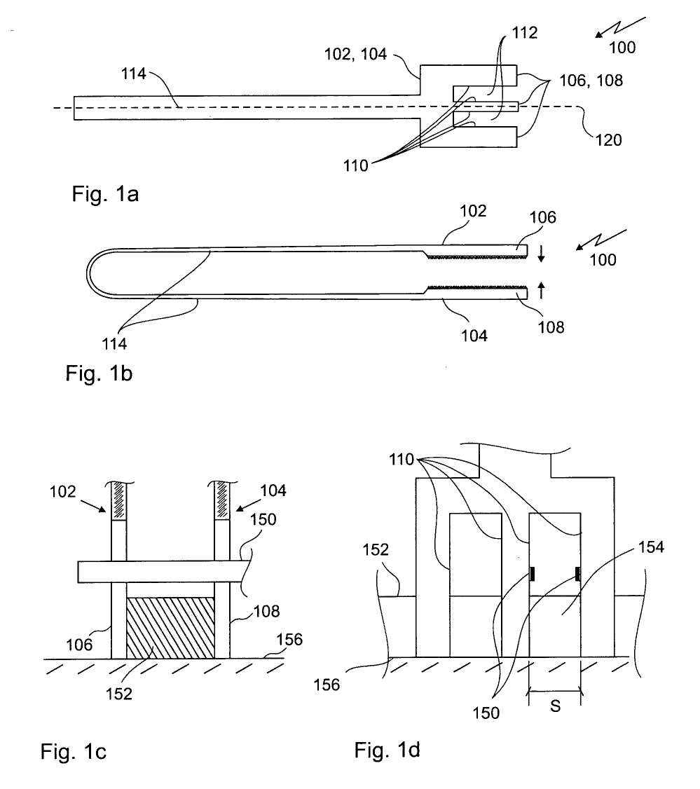

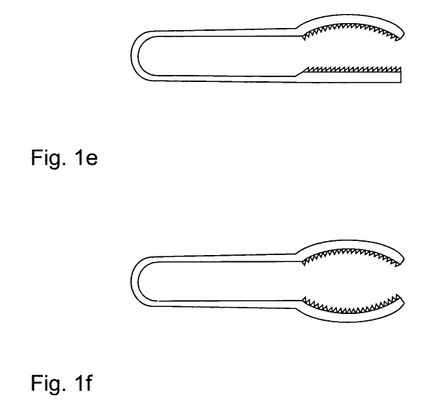

Referring to FIGS. 1a and 1b, a side view and a top view of a grossing tool 100 according to the invention is shown. The grossing tool 100 comprises a first jaw 102 and a second jaw 104 for holding a tissue sample in a substantially fixed position therebetween. The first jaw 102 and the second jaw 104 comprise a first array of prongs 106 and a second array of prongs 108, respectively, for engaging the tissue sample. The prongs of each of the first array of prongs 106 and the second array of prongs 108 have parallel edges for guiding a cutting tool 150 such as a thin knife or scalpel, as shown in a cross sectional view in FIG. 1c and in a side view in FIG. 1d. The parallel edges 110 form at least a cutting slot 112 of a predetermined width S corresponding to a predetermined thickness of a slice 154 to be cut from the tissue sample 152. The at least a cutting slot 112 of the first array of prongs 106 are placed opposite the corresponding at least a cutting slot 112 of the second array of prongs 108. Thus, the cutting tool 150 is guided by the edge of a prong of the first array 106 and a corresponding edge of a prong of the second array, enabling a uniform cut. Repeating the cutting along the edges on the opposite side of the cutting slot 112 provides the slice 154 having a substantially uniform thickness corresponding to the width S of the cutting slot 112. The grossing tool further comprises an actuator 114 in mechanical communication with the first jaw 102 and the second jaw 104. The actuator 114 enables, preferably manual, movement of the first jaw 102 and the second jaw 104 in a plane of movement 120, which is oriented perpendicular to the first array of prongs 106 and the second array of prongs 108, between an open position and a holding position for holding the tissue sample 152. In operation, a user holds the actuator 114 and grasps the tissue sample 152 between the first array of prongs 106 and the second array of prongs 108 by moving the first jaw 102 and the second jaw 104 towards each other until the tissue sample 152 is hold in a substantially fixed position. Optionally, the grasping is facilitated by disposing the tissue sample 152 onto a plane surface 156 and sliding the prongs with their tips along the surface 156 towards the tissue sample 152. The tissue sample 152 is then held in a substantially fixed position between the first array of prongs 106 and the second array of prongs 108 with the tips of the prongs in contact with the surface 156. The slice 154 of the tissue sample 152 is then cut by moving a cutting tool 150 towards the surface 156 and guided by the edges 110 of the prongs on a first side of the cutting slot 112 for a first cut and on a second opposite side of the cutting slot 112 for a second cut resulting in the slice 154 having a predetermined and substantially uniform thickness S. Further optionally, one of the first jaw 102 and the second jaw 104, shown in FIG. 1e, comprises a concave curved surface facing the opposite jaw or, as shown in FIG. 1f, both jaws comprise a concave curved surface facing the opposite jaw—with both jaws having a same curvature or, alternatively, a different curvature—in order to facilitate holding of curved samples such as a prostate. As is evident, the grossing tool 100 is highly beneficial for cutting tissue sample slices. For example, it enables even an inexperienced user to cut tissue samples having a predetermined and substantially uniform thickness. By engaging the tissue sample between the first and the second array of prongs even tissue samples that are very soft, smooth and slippery are held in a substantially fixed position without substantially distorting the tissue, further contributing to the uniformity of the slice thickness. Furthermore, the simple structure of the grossing tool provides easy handling and cleaning as well as improved portability.

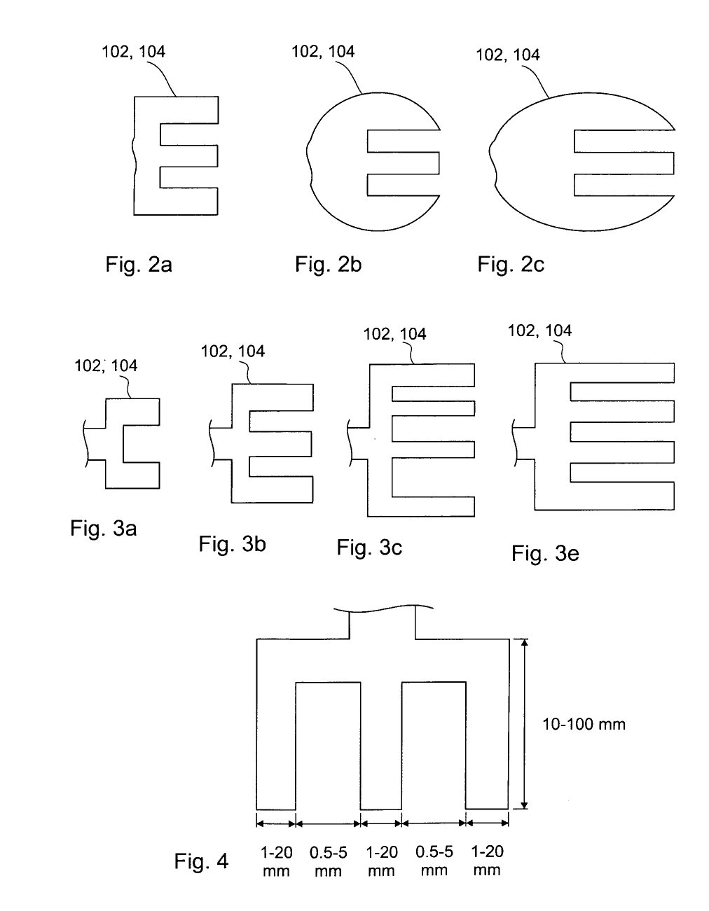

The jaws 102, 104 are, for example, easily made of a plate of approximately 1 mm to 8 mm thickness having the portion representing the at least a cutting slot 112 removed. The plate is, for example, made of metal—preferably stainless steel, plastic, non-sticking plastic coated metal or carbon fiber. The slots are cut out from an initial plate using, for example, a laser cutting process. Referring to FIGS. 2a to 2c various examples of different shapes of the plates for use in the grossing tool 100 are shown such as a rectangular shape—FIG. 2a, round shape—FIG. 2b, and ovoid shape—FIG. 2c.

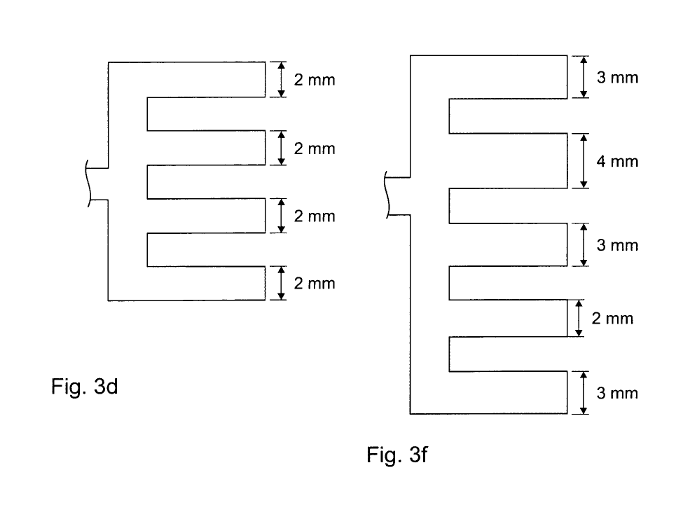

FIGS. 3a to 3f illustrate various different embodiments of jaws for use in the grossing tool 100. In its simplest form the jaws 102, 104 each comprise two prongs defining a single cutting slot, as shown in FIG. 3a. FIG. 3b shows a jaw 102, 104 having two cutting slots of a same width enabling a user to cut two slices in a single process without removing and grasping again the tissue sample. The jaw 102, 104 shown in FIG. 3c comprises cutting slots of different width enabling cutting of slices having a different thickness using a single grossing tool which is, for example, beneficial for grossing off site by reducing the number of tools needed for preparing sample slices of various thicknesses. As shown in FIG. 3d, all prongs have a same width, for example, 2 mm. Alternatively, in order to provide a better grip, the jaws 102, 104 comprise outer prongs having greater width than inner prongs, as shown in FIG. 3e, which is advantageous for gripping, for example, samples of fatty tissue. Further alternatively, the width of the prongs between the first and the last prong is varied between 1 mm and 5 mm, as shown, for example, in the five prong design in FIG. 3f with the prong width varying from 2 mm to 4 mm. As is evident, there are numerous different designs of the jaws 102, 104 possible having various numbers of prongs with, for example, different dimensions. Obviously, the prongs and cutting slots are easily dimensioned according to various specific needs. For example, for preparing tissue sample slices for histoprocessing the width of the cutting slots is varied between 0.5 mm and 5 mm—with 1.5 mm being the most preferred, the width of the prongs is varied between 1 mm and 20 mm—with 2 mm being the most preferred for inner prongs and 3 mm for outer prongs, and the length of the prongs is varied between 10 mm and 100 mm, as shown in FIG. 4.

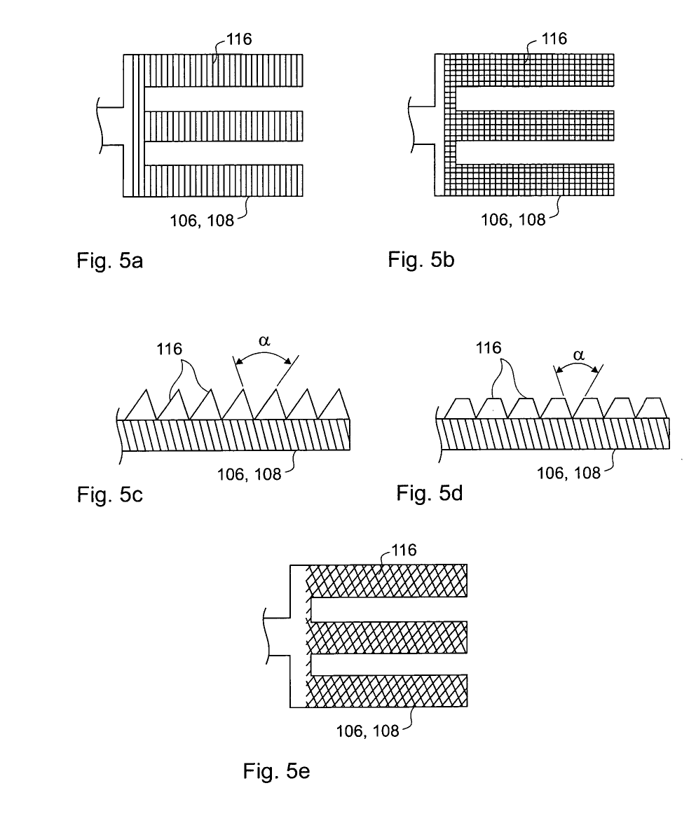

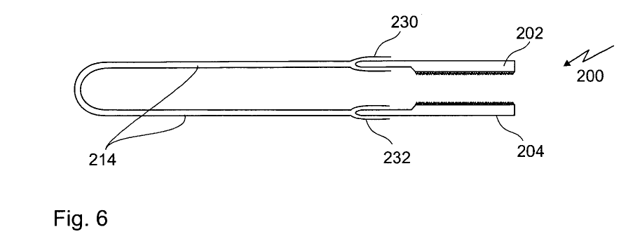

To improve engagement of the prongs with the tissue sample a gripping member 116 is disposed on a surface of each prong facing a corresponding opposite prong. The gripping member comprises, for example, a grooved surface such as V-grooves, as shown in FIG. 5a, or a toothed surface, as shown in FIG. 5b. The toothed surface comprises teeth of, for example, pyramidal shape, as shown in FIG. 5c, or pyramidal shape with flattened apex, as shown in FIG. 5d. For example, the teeth are dimensioned according to various specific needs with the teeth having a height between 0.2 mm and 3 mm, a base width between 1 mm and 3 mm, and an angle α between 20° and 100°. The teeth are arranged in rows parallel to the edges of the prongs, as shown in FIG. 5b, or at an angle to the edges of the prongs, as shown in FIG. 5e. Of course, it is possible to employ numerous other shapes and arrangements of grooves or teeth.

In another embodiment 200 of the grossing tool, jaws 202 and 204 are removably attached to actuator 214 via attachment members 230 and 232, respectively. There are numerous ways to removably attach the jaws 202 and 204 using, for example, frictional coupling, a securing pin, or a securing screw. Removably attaching the jaws 202 and 204 allows, for example, use of disposable jaws for reducing the risk of cross contamination or, alternatively, use of a single actuator in combination with jaws having various different cutting slot widths. It also enables easy replacement of the jaws 202 and 204 due to wear, in particular, when the jaws are made of a plastic material or carbon fiber material, while retaining the actuator 214.

As is evident, there are numerous possibilities for realizing an actuator for moving and holding the jaws. For example, in the embodiment 100 shown in FIGS. 1a and 1b the actuator 114 comprises a U-shaped band made of an elastic material such as metal, high density plastic, or carbon fiber having at an end of each arm a jaw 102, 104. The U-shaped band acts as a compression spring when the first jaw 102 and the second jaw 104 are moved from the open position to the holding position. This is the simplest for of an actuator enabling a single piece design of the grossing tool 100 by forming the ends of the U-shaped band into the first 102 and the second jaw 104. Furthermore, due to its simplicity it is the easiest to clean as well as the cheapest to manufacture allowing, for example, manufacturing of a disposable grossing tool made of a relatively cheap plastic material.

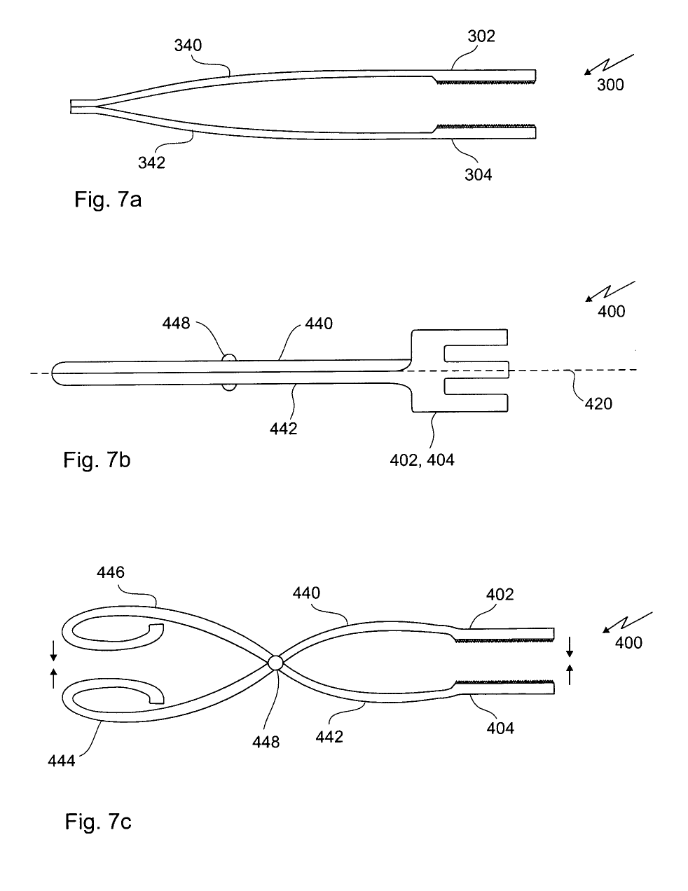

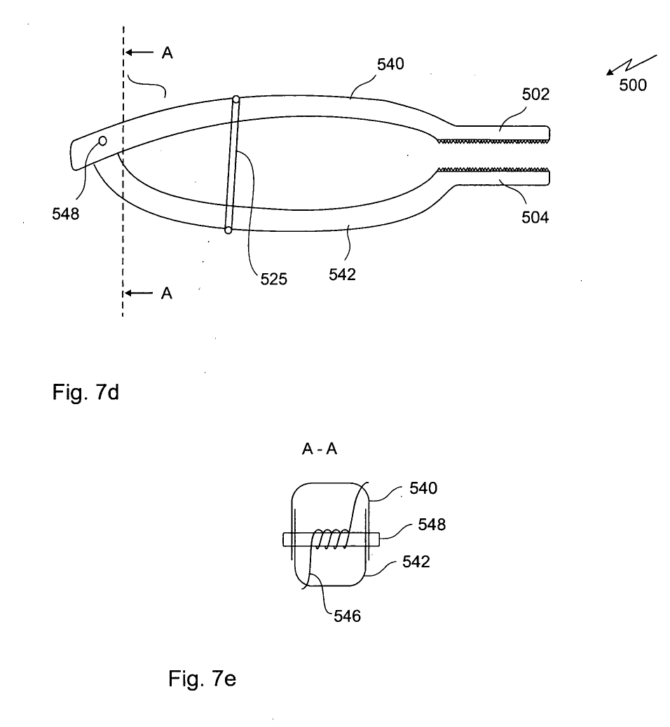

Referring now to FIG. 7a an embodiment 300 of a grossing tool according to the invention is shown. Here the U-shaped band is replaced by two arms 340 and 342, respectively, made again of an elastic material. The two arms 340 and 342 are each connected at a first end to ajaw 302, 304 and connected with each other at a second opposite end. As above, the connected two arms act as a compression spring when the first jaw 302 and the second jaw 304 are moved from the open position to the holding position. Optionally, each of the arms 340 and 342 together with the jaws 302 and 304, respectively, are made as a single piece.

Referring to FIGS. 7b and 7c, a side view and a top view of an embodiment 400 of a grossing tool according to the invention is shown. The grossing tool comprises a first member 440 having at a first end a jaw 402 and at a second opposite end a first handle 444, and a second member 442 having at a first end a jaw 404 and at a second opposite end a second handle 446. A pivot 448 disposed between the jaws 402, 404 and the handles 444, 446 connects the first member 440 and the second member 442 and allows pivotally moving the first jaw 402 and the second jaw 404 in a plane of movement 420 between the open position and the holding position by manually moving the handles 444 and 446 in a scissor-like action. The first member 440 and the second member 442 are made of, for example, metal, plastic, or carbon fiber material. Optionally, each of the first member 440 and the second member 442 together with the jaws 402 and 404, respectively, are made as a single piece.

Referring to FIGS. 7d and 7e, a top view and a cross-sectional view A-A of an embodiment 500 of a grossing tool according to the invention is shown. The grossing tool comprises a first member 540 having at a first end ajaw 502 and a second member 542 having at a first end a jaw 504. A pivot 548 connects the first member 540 and the second member 542 at a second opposite end and allows pivotally moving the first jaw 502 and the second jaw 504 between the open position and the holding position by manually moving the first member 540 and the second member 542. The first member 540 and the second member 542 are, preferably, made of a rigid material, for example, metal, plastic, or carbon fiber material. The cross-sectional view in FIG. 7e shows the first member 540 and the second member 542 having a U-shaped cross-section with the second member 542 fitting into the U-shaped first member at the location of the pivot 548. Furthermore, the member 540 and the second member 542 are spring loaded using tension spring 546 surrounding the pivot 548 and acting on the first member 540 at a first end and on the second member 542 at a second end. Optionally, a securing ring 525 is disposed around the member 540 and the second member 542 for keeping the same in a fixed position, for example, the holding position. Further optionally, a groove-like indentation is provided on the first member 540 and the second member 542 in order to enable placement of the securing ring 525 at a predetermined location with respect to the first member 540 and the second member 542.

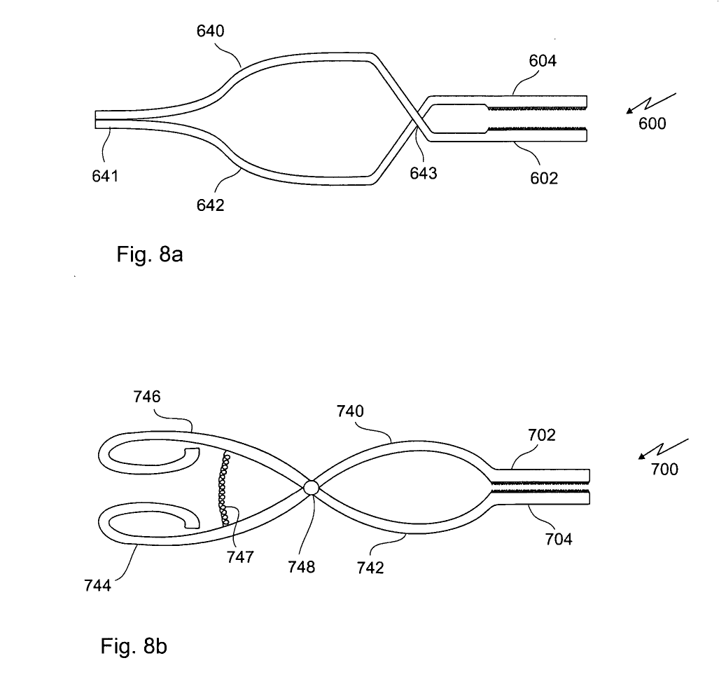

Referring to FIGS. 8a and 8b, two further embodiments 600 and 700 of a grossing tool according to the invention are shown. The embodiments 600 and 700 comprise a pre-stress mechanism providing a predetermined stress for maintaining the first jaw and the second jaw in the holding position absent user interaction. Here the user acts against the pre-stress mechanism in order to open the jaws. The tissue sample is then grasped and hold in a substantially fixed position due to action of the pre-stress mechanism. Providing a pre-stress mechanism has two major advantages. First, maximum pressure acting on the tissue sample is predetermined and limited by the pre-stress mechanism and, therefore, not depending on the manual user action. Second, it frees the user's hand from having to act, for example, pressing the two arms or handles together while cutting. This allows a more rested positioning of the user's hand enabling a more precise cut.

In the embodiment 600 shown in FIG. 8a the pre-stress mechanism is realized by crossing the two arms 640 and 642 over each other in a cross-over section 643. The compression spring like action of the arms 640 and 642 maintains the jaws 602 and 604 in the holding position without user interaction. The jaws are opened by pressing the two arms towards each other between their connection 641 and the cross-over section 643.

In the alternative embodiment 700 the pre-stress mechanism is realized by interposing a tension spring 747 between pivot 748 and the handles 744 and 746. The tension spring acting on the members 740 and 742 maintains the jaws 702 and 704 in the holding position without user interaction. Here the jaws are opened by moving the handles 744 and 746 outwardly acting against the tension spring. Alternatively, the tension spring 747 is interposed between the pivot 748 and the jaws 702, 704.

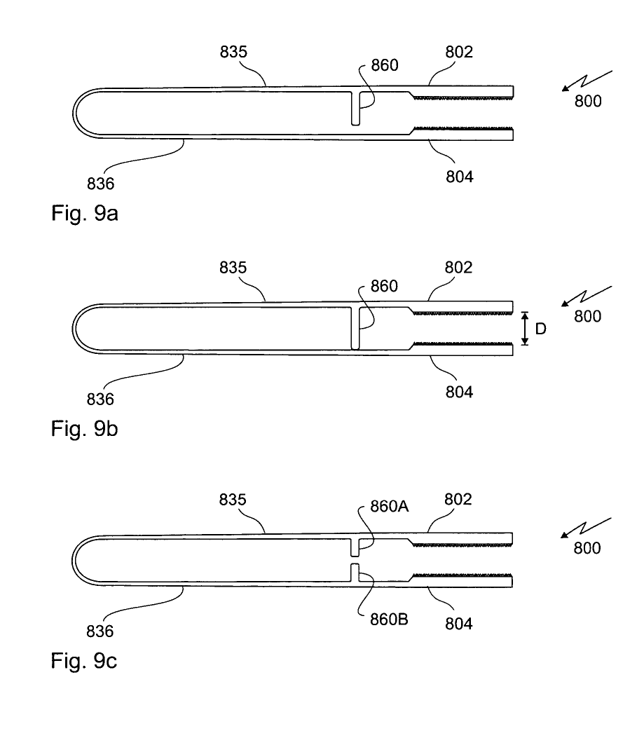

In the embodiment 800, shown in FIGS. 9a to 9c, the grossing tool according to the invention comprises a stopper 860 interposed between the first arm 835 and the second arm 836 and connected to the first 835 arm, as shown in FIG. 7a. In the holding position the stopper 860 abuts the second arm 836 stopping movement of the first jaw 802 and the second jaw 804 at a predetermined distance D therebetween, as shown in FIG. 9b. Providing a stopper reduces the risk of squeezing the tissue sample between the jaws, for example, when an inexperienced user provides too much pressure to the actuator while holding the tissue sample. Alternatively, the stopper comprises two sections 860A and 860B connected to the first arm 835 and to the second arm 836, respectively, as shown in FIG. 9c. In the holding position the two stopper sections 860A and 860B abut each other. Obviously, the stop mechanism is realized in the above embodiments in a similar fashion.

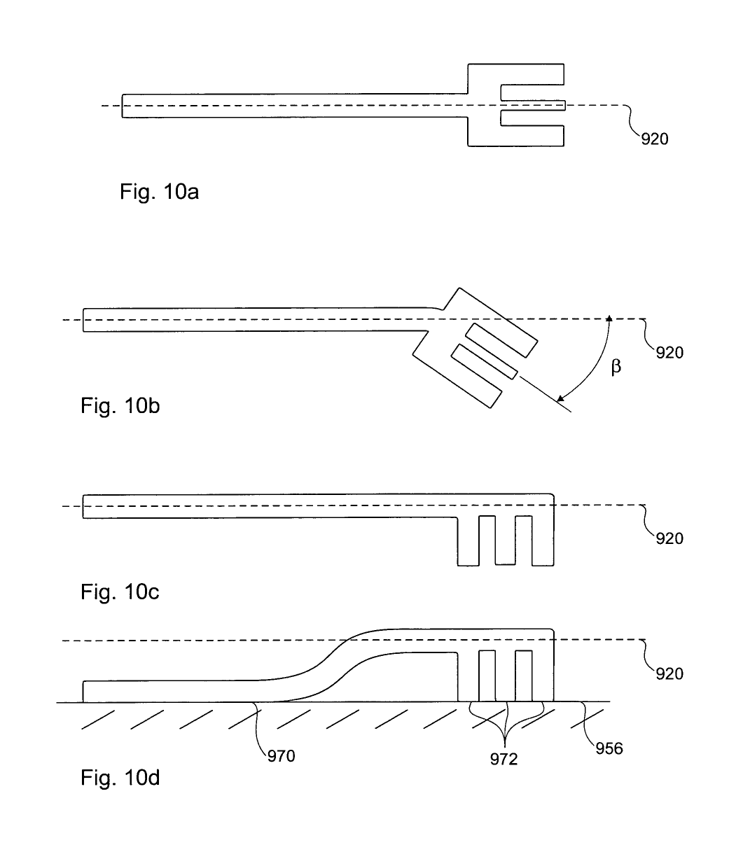

According to user requirements and to provide ergonomically improved tools the prongs are oriented at various angles to the plane of movement 920, as shown in FIGS. 10a to 10d. In the embodiment shown in FIG. 10a the prongs are oriented parallel to the plane of movement. In ergonomically improved tools the prongs are oriented at an angleβ, as shown in FIG. 10b, with angles between 20° and 70° being the most preferred ones. In the embodiment shown in FIG. 10c the prongs are oriented perpendicular to the plane of movement 920. Finally, in the embodiment shown in FIG. 10d the arms of the actuator comprise a straight edge 970 which are disposed such that the straight edge 970 of each arm and the tip 972 of each prong are located in a same plane parallel to the plane of movement 920. This allows holding of the actuator as well as the tips of the prongs against a plane surface 956, allowing resting of a user's hand on the plane surface 956 while holding the actuator and cutting a sample slice, thus enabling a more precise cut. Evidently, the various orientations of the prongs as illustrated in FIGS. 10a to 10d are realized with any of the above embodiments.

Obviously, the various embodiments of the grossing tool according to the invention outlined above are easily combined in various fashions in order to satisfy specific needs in different applications.

Numerous other embodiments of the invention will be apparent to persons skilled in the art without departing from the spirit and scope of the invention as defined in the appended claims.

What is claimed is:

1. A grossing tool comprising:
a first and a second jaw for holding a tissue sample in a substantially fixed position therebetween, the first jaw and the second jaw having a first and a second array of prongs, respectively, for engaging the tissue sample, the prongs of each of the first and the second array of prongs having parallel edges for guiding a cutting tool, the parallel edges forming at least a cutting slot of a predetermined width corresponding to a predetermined thickness of a slice to be cut from the tissue sample, the at least a cutting slot of the first array of prongs being placed opposite the corresponding at least a cutting slot of the second array of prongs; and,
an actuator in mechanical communication with the first jaw and the second jaw for moving the first jaw and the second jaw in a plane of movement oriented perpendicular to the first and the second array of prongs between an open position and a holding position for holding the tissue sample.

2. A grossing tool as defined in claim 1 wherein at least one of the first and the second jaw comprises a concave curved surface facing the opposite jaw.

3. A grossing tool as defined in claim 1 wherein each of the first and the second jaw comprises a plate having at least a portion representing the at least a cutting slot removed.

4. A grossing tool as defined in claim 2 wherein the plate has one of rectangular, round, and ovoid shape.

5. A grossing tool as defined in claim 2 wherein the plate is made of one of metal, plastic, non-stick plastic coated metal and carbon fiber.

6. A grossing tool as defined in claim 1 wherein each of the first and the second array of prongs form a plurality of cutting slots having a same width.

7. A grossing tool as defined in claim 1 wherein each of the first and the second array of prongs form a plurality of cutting slots having different width.

8. A grossing tool as defined in claim 1 wherein a first and a last prong of the first and the second array have a greater width than prongs therebetween.

9. A grossing tool as defined in claim 1 wherein all prongs of the first and the second array have a same width.

10. A grossing tool as defined in claim 1 wherein the predetermined width of the at least a cutting slot is between 0.5 mm and 5 mm.

11. A grossing tool as defined in claim 10 wherein the prongs have a width between 1 mm and 20 mm.

12. A grossing tool as defined in claim 11 wherein the prongs between a first prong and a last prong of the first and the second array have a width between 1 mm and 5 mm and wherein different prongs of each of the first and the second array have a different width.

13. A grossing tool as defined in claim 10 wherein the prongs have a length between 10 mm and 100 mm.

14. A grossing tool as defined in claim 1 wherein the prongs comprise a gripping member on a surface facing a corresponding prong of the opposite array of prongs.

15. A grossing tool as defined in claim 14 wherein the gripping member comprises a grooved surface.

16. A grossing tool as defined in claim 14 wherein the gripping member comprises a toothed surface.

17. A grossing tool as defined in claim 1 wherein the first jaw and the second jaw are removably attached to the actuator.

18. A grossing tool as defined in claim 1 wherein the actuator comprises:
a first arm connected at a first end to the first jaw; and,
a second arm connected at a first end to the second jaw.

19. A grossing tool as defined in claim 18 wherein the first arm and the second arm are made of an elastic material and are joined at a second opposite end, the first arm and the second arm acting as a compression spring when the first jaw and the second jaw are moved from the open position to the holding position.

20. A grossing tool as defined in claim 18 wherein the first arm and the second arm are connected in a pivot, the first arm and the second arm each comprising a handle portion extending from the pivot opposite the first jaw and the second jaw, respectively, for moving the first jaw and the second jaw between the open position and the holding position.

21. A grossing tool as defined in claim 18 comprising a pre-stress mechanism in mechanical communication with the first and the second arm, the pre-stress mechanism providing a predetermined stress for maintaining the first jaw and the second jaw in the holding position absent user interaction.

22. A grossing tool as defined in claim 21 wherein the first arm and the second arm are made of an elastic material and are joined at a second opposite end, the first arm and the second arm acting as a compression spring, and wherein at least one of the first arm and the second arm comprises a cross-over section for crossing over the other arm for providing a predetermined stress for maintaining the first jaw and the second jaw in the holding position absent user interaction.

23. A grossing tool as defined in claim 18 comprising a stop mechanism interposed between the first and the second arm for stopping movement of the first jaw and the second jaw in the holding position with the first jaw and the second jaw being stopped having a predetermined distance therebetween.

24. A grossing tool as defined in claim 23 wherein the stop mechanism comprises a stopper attached to the first arm, the stopper abutting the second arm in the holding position.

25. A grossing tool as defined in claim 18 wherein the first arm and the second arm are connected in a pivot at a second opposite end.

26. A grossing tool as defined in claim 25 comprising a tension spring disposed around the pivot and in mechanical communication with the first arm and the second arm for maintaining the first jaw and the second jaw in an open position.

27. A grossing tool as defined in claim 25 wherein the first arm and the second arm are made of a rigid material.

28. A grossing tool as defined in claim 18 comprising a securing ring disposed around the first arm and the second arm for maintaining the first arm and the second arm in a fixed position relative to each other.

29. A grossing tool as defined in claim 18 wherein the prongs are oriented parallel to the plane of movement.

30. A grossing tool as defined in claim 18 wherein the prongs are oriented at an angle to the plane of movement.

31. A grossing tool as defined in claim 30 wherein the prongs are oriented perpendicular to the plane of movement.

32. A grossing tool as defined in claim 30 wherein at least a portion of each of the first and the second arm comprises a straight edge such that the straight edge and a tip of each of the prongs are disposed in a same plane oriented parallel to the plane of movement.

33. A grossing tool comprising: a U-shaped band of elastic material having at a first end a first jaw and at a second end a second jaw for holding a tissue sample in a substantially fixed position therebetween, the first jaw and the second jaw having a first and a second array of prongs, respectively, for engaging the tissue sample, the prongs of each of the first and the second array of prongs having parallel edges for guiding a cutting tool, the parallel edges forming at least a cutting slot of a predetermined width corresponding to a predetermined thickness of a slice to be cut from the tissue sample, the at least a cutting slot of the first array of prongs being placed opposite the corresponding at least a cutting slot of the second array of prongs, the first jaw and the second jaw being movable in a plane of movement oriented perpendicular to the first and the second array of prongs between an open position and a holding position for holding the tissue sample.

34. A grossing tool as defined in claim 33 wherein the grossing tool is made as a single piece.

35. A grossing tool as defined in claim 34 wherein the U-shaped band acts as a compression spring when the first jaw and the second jaw are moved from the open position to the holding position.

36. A grossing tool comprising:
a first member having at a first end a first jaw and at a second opposite end a first handle and a second member having at a first end a second jaw and at a second opposite end a second handle, the first jaw and the second jaw for holding a tissue sample in a substantially fixed position therebetween, the first and the second jaw having a first and a second array of prongs,
respectively, for engaging the tissue sample, the prongs of each of the first and the second array of prongs having parallel edges for guiding a cutting tool, the parallel edges forming at least a cutting slot of a predetermined width corresponding to a predetermined thickness of a slice to be cut from the tissue sample, the at least a cutting slot of the first array of prongs being placed opposite the corresponding at least a cutting slot of the second array of prongs; and,
a pivot for connecting the first member and the second member and for moving the first jaw and the second jaw in a plane of movement oriented perpendicular to the first and the second array of prongs between an open position and a holding position for holding the tissue sample.

37. A grossing tool as defined in claim 36 wherein the first member and the second member are each made as a single piece.

38. A grossing tool as defined in claim 37 wherein the first member and the second are connected between the first jaw and the first handle and between the second jaw and the second handle, respectively.
